(19) United States
(12) Patent Application Publication
(10) Pub. No.: US 2003/0152962 A1
(43) Pub. Date: Aug. 14, 2003

(54) Detection of nerve tissue damage

(76) Inventor: Jack Price, London (GB)

Correspondence Address: BANNER & WITCOFF, 1001 G STREET N W, SUITE 1100, WASHINGTON, DC, 20001, US

(21) Appl. No.: 10/240,170

(22) PCT Filed: Mar. 30, 2001
(86) PCT No.: PCT/GB01/01442

(30) Foreign Application Priority Data
Mar. 30, 2000 (GB) 0007778.4

(51) Int. Cl. C12Q 1/68; G01N 33/53; G01N 33/567

(52) U.S. Cl. 435/6; 435/7.2

(57) ABSTRACT

This invention relates to diagnostics in the fields of neurotoxicology and neuropathology and more particularly to the visualisation of areas of damage to nerve tissue. In particular, the present invention relates to the use SCIP as a marker of neurological damage.

This invention relates to diagnostics in the fields of neurotoxicology and neuropathology and more particularly to the visualisation of areas of damage to nerve cells and/or tissue.

The detection of damage to nerve cells and/or tissue is important when testing for the toxicity of drugs (i.e. determining the neurotoxicology of drugs) and when determining the presence of a neuropathology.

In the toxicity testing of drugs it is necessary to determine whether the test compound has any adverse effects on the central nervous system. This determination has a number of components: First is the question whether the compound crosses the blood-brain barrier and, if so, whether it has any toxic effects; second it must be determined where in the brain or central nervous system any toxic effects are localised; third, what doses of the compound give the effects and what doses are safe?

Studies on nerve cells in culture can give some generalised data on toxicity and dose effects, but conventionally these questions are addressed using behavioural studies, often in the form of an Irwin profile. Ascending doses of the compound are injected into animals, which are then observed and assessed over a range of parameters relating to feeding, sleep, movement, etc. These assays have the disadvantage of being slow, resource-intensive, and difficult to interpret.

The problem of determining the presence of a neuropathology is how to recognise areas of brain damage or disease where specific markers of damage may not be available. Some disorders are characterised by very specific pathological features. Examples are the phosphorylated Tau and neurofibrillary tangles of Alzheimer's disease, and the depleted dopaminergic neurons of Parkinson's disease. Many disorders, however, have no such markers, and consequently have been difficult to define. An example of this type of disorder is Frontal lobe dementia, which is responsible for probably 10% of all dementias (compared to 40% for Alzheimer's disease) but hardly registers as a disorder because the pathology is ill-defined. Another example is that of Schizophrenia, where there is almost certainly some neuropathology, but it is too ill-defined and difficult to recognise to be a useful criterion.

Schizophrenia is a brain disease whose aetiology is largely unknown, but one current hypothesis is that the origins of the disorder lie early in life, possibly during prenatal brain development. This ‘neurodevelopmental hypothesis’ suggests that a brain abnormality is present early in life but does not fully manifest itself clinically until late adolescence or early adulthood. This hypothesis has grown from studies of the neuropathology and epidemiology of the disease, and has been supported by more recent imaging studies. These latter studies have demonstrated an enlargement of the cerebral ventricles in schizophrenic patients as well as structural abnormalities in the frontal and temporal lobes. This agrees, in general, with neuropathological reports of temporal and frontal lobe abnormalities of the schizophrenic brain. Pathological studies also indicate that subtle abnormalities of cortical development may be present. The findings of cytoarchitectural abnormalities, along with a lack of gliosis, have been taken as evidence that schizophrenia is a developmental disorder. Nonetheless, the pathological findings have been distinguished mostly by their variability, and by the subtlety of the changes observed in schizophrenic patients in the markers that have been described.

In studies of the expression of POU domain transcription factors during brain development it has been found that a particular transcription factor, called SCIP (suppressed cAMP inducible POU) and also known as Oct-6 and Tst-1, is expressed in certain populations of brain cells during development. SCIP appears to have a predominant developmental role being expressed in embryonic stem (ES) cells, and the mouse inner cell mass (Suzuki et al., EMBO, 1990; 2: 3723-3732 and Meijer et al., Nucleic Acids Res., 1990; 18: 7357-65), but its best-characterised role is in Schwann cell development in the peripheral nervous system where it regulates the timely onset of myelination (Bermingham et al., Genes Dev., 1996;15:1751-62).

In the developing rodent telencephalon, SCIP expression is turned on as neurons become post-mitotic and migrate to their final positions in the cortical plate, the embryonic cortical grey matter. This means that SCIP is expressed during the period in which neurons first begin to establish their neuronal identity and axonal projection, and while they find their definitive cortical layer. In the postnatal brain, SCIP expression is mostly lost, but is retained by certain specific sub-populations of neurons in layer 5 and 2/3 of the cerebral cortex, and CA1 field of the hippocampus (Frantz et al., J. Neurosci., 1994; 14: 472-485). The role of SCIP in neuronal development is unknown, but the timing of its expression suggests that it may play a role in establishing neuronal sub-type identity.

It has now been discovered that normal adult brain expresses minimal levels of SCIP protein, but if the brain has been damaged, then SCIP is expressed at significant levels by nerve cells at the sites of damage. This appears to be true whatever the nature of the damaging agent. This phenomenon has been demonstrated, for example, in human brain damaged by focal cortical dysplasia and schizophrenia, and in rodent brain damaged by physical injury, epileptic electrical activity, or by ischaemia. SCIP can therefore be use as a marker of nerve tissue damage. Moreover, SCIP expression appears to be stable. Once SCIP is turned on in response to damage, it remains expressed for many months or even years.

There is a need in the art for a method for quickly and easily determining the neurotoxicity of drugs and for determining the presence of neurological damage, especially neurological damage for which no marker has been defined.

The present invention provides the use of SCIP as a marker of neurological damage.

The present invention provides a method of detecting neurological damage comprising assaying for the expression of a SCIP gene in nerve cells and/or tissue in which expression of increased levels of SCIP indicates neurological damage.

It has been found that adult nerve cells and/or tissue, especially brain, expresses minimal levels of SCIP protein, but if the nerve cells and/or tissue has been damaged, then SCIP is expressed at increased levels by nerve cells at the site of damage irrespective of the nature of the damaging agent. Increased levels are levels which result in, the easy detection of SCIP encoding mRNA or SCIP protein using standard assay techniques such as in situ hybridisation using a labelled polynucleotide or immunohistochermistry using labelled antibody molecules. Preferably, the level of SCIP expression, as measured by the level of mRNA or SCIP protein is increased at least 50%, more preferably at least 100% compared to the level in corresponding nerve cells and/or tissue that has not been damaged. Accordingly, by assaying for the expression of the SCIP gene in nerve tissue it is possible to determine whether there has been any neurological damage.

The term “neurological damage” refers to any damage of the nervous system including the brain and the central nervous system. Preferably the term means any damage to the brain. The damage may be caused by accident or by a disease including damage generated by physical injury, ischaemic insult, developmental injury, or acute neurotoxic insult. Examples of neurological damage include cytotoxic damage of neurones leading to neuronal loss; damage to axons or dendritic processes leading to loss of neuronal projections and demyelination; inflammation of the nervous system leading to glial proliferation, scarring, and cytotoxic responses. Further examples of neurological damage include psychiatric or neurodegenerative disorders such as schizophrenia or frontal lobe dementia and epilepsy. The neurological damage may also be within an animal wherein the damage has been purposefully induced, for example in a toxicology study involving injection of a potentially toxic drug.

The term “SCIP gene” refers to the human, mouse, rat, or any other functionally equivalent homolog or mutant of the SCIP gene. The sequence of the human SCIP gene has accession number NM 002699 (Genebank) and is described in Monuki et al, Science, 249, 1300-1309, 1990. The rat SCIP gene has accession number M72711 (Genebank) and is described in Kuhn et al, Mol. Cell. Biol, 11, 4642-4650, 1991. The sequence of the mouse SCIP gene has accession number M88302 (Genebank) and is described in Hara et al, PNAS USA, 89, 3280-3284, 1992. There is great homology between the SCIP genes of human and rodents, with the human sequence being 98.8% homologous to the mouse and rat sequence.

The term “functionally equivalent homologs and mutants of a native SCIP gene” refers to any nucleotide sequence which has at least 80% sequence homology with the sequence of the human SCIP gene and which is expressed at sites of neurological damage. Preferably the SCIP gene has at least 90% sequence homology with the human SCIP gene and is expressed at sites of neurological damage.

The term “SCIP protein” as used herein refers to any polypeptide encoded by SCIP gene as defined above and includes proteins which have post-translation modifications such as the addition of carbohydrate groups.

The term “SCIP mRNA” as used herein refers to any mRNA transcribed from the SCIP gene as defined above and includes truncated mRNA transcripts and alternatively spliced mRNA transcript.

The expression of the SCIP gene may be assayed by using any suitable assay procedure. Preferably, expression of the SCIP gene is assayed using an antibody molecule having affinity for the SCIP protein encoded by the SCIP gene. Alternatively, a probe, such as a labelled polynucleotide probe, can be used to identify the presence of SCIP encoding mRNA. As will be apparent to those skilled in the art, there are numerous other methods such as RT PCR which can be used to detect SCIP mRNA.

The nerve tissue can be any nerve tissue including the brain and central nervous system and the nerve cells can be derived from any nerve tissue. Preferably the nerve tissue is brain, more preferably the nerve tissue is the cerebral cortex of a brain.

In a particular preferred embodiment, the method of the present invention comprises obtaining a sample of nerve cells and/or tissue from a subject and contacting the nerve cells and/or tissue with an antibody molecule having affinity for SCIP protein in order to determine if SCIP protein is present.

The antibody molecule may be any antibody molecule which is capable of specifically binding the SCIP protein. The antibody molecule may be a polyclonal antibody or a monoclonal antibody. Fragments of antibodies capable of specifically binding the SCIP protein may also be used, such as Fv, Fab, F(ab′)₂ fragments and single chain Fv fragments. The antibody molecule may be a recombinant antibody molecule such as a chimeric antibody molecule. Methods for producing such antibody molecules are well known to those skilled in the art.

The antibody molecule is preferably labelled. Suitable labels include horseradish peroxidase (HRP), chloramphenicoltransferase (CAT), digoxygenin (DIG), fluorescein and radioisotopes such as ¹²⁵I, ³H and ¹⁴C.

Depending on the label used, the amount of labelled antibody molecule immobilised can be determined using standard methods well known to those skilled in the art. For example, if the label is HRP, the degradation of luminol by the enzyme and the associated emission of chemiluminescence can be measured. However, if a radioactive label is used, the presence of the label is measured by detecting the emitted radiation.

It is also possible to provide a first antibody molecule having affinity for SCIP protein and a second labelled antibody molecule having affinity for the first antibody molecule. The use of such combinations of antibody molecules is well known to those skilled in the art.

The method of the present invention may also be performed wherein a sample of nerve cells and/or tissue is obtained from a subject and contacted with a probe that recognises SCIP mRNA.

Preferably the probe is labelled. Suitable labels include any one of the labels referred to above with respect to the antibody molecule. Preferably the probe is labelled with digoxygenin and is detected by using an anti-dioxygenin antibody conjugated to alkaline phosphatase. Such antibodies are available from Boehringer Mannheim. Preferably the probe is a nucleic acid probe such as an RNA probe or DNA probe.

The probe is preferably a nucleic acid probe having a sequence corresponding to that of at least part of the SCIP mRNA. The probe may be of any size; however, preferably the probe is about 10 to 500, more preferably about 20 to 300 and most preferably about 30 to 200 nucleotides in length.

It is preferred that the sequence of the probe corresponds to any part of the SCIP mRNA which is unique to the SCIP gene. Accordingly, it is preferred that the probe does not have a sequence corresponding to the POU homeo-domain or the POU-domain. The POU homeo-domain and the POU-domain are well defined and known to those skilled in the art. For example, the POU homeo-domain of the mouse SCIP gene encodes amino acids 335 to 396 of the mouse SCIP protein and the POU-domain of the mouse SCIP gene encodes amino acids 240 to 319 of the mouse SCIP protein. The POU homeo-domain and POU-domain of the human and rat SCIP gene are in substantially the same positions as in the mouse SCIP gene.

Preferably the probe is a nucleic acid probe corresponding to part of the SCIP mRNA encoding the N-terminal region of the SCIP protein. Preferably the probe is an RNA probe produced by transcribing the following sequence using T3 and T7 polymerases. 5′ggaggcggcggcgcgggacccggctgcaccacgcactgcacgaggacggccacgaggcacagctggagccgtcgccaccaccgcacctgggcgcacacggacacgcacggacatgcacacgcgggcggcctgcacgcggcggcggcggcgcacctgcaccggg3′

The invention provides a means of identifying areas of nerve cell and/or tissue damage by using a reagent that recognises either the SCIP protein or the mRNA transcribed from the SCIP gene.

The nerve cells and/or tissue under consideration may be removed from a subject suspected of harbouring neurological damage. The nerve cells and/or tissue may be removed post-mortem or removed while the subject is alive as a biopsy. The subject may be a human or a non-human animal such as a mouse or a rat.

Nerve tissue can be prepared for conventional immunohistochemistry, using standard procedures known to those practiced in the art. For example, when the nerve tissue is brain, the brain is fixed in a standard fixative, such as formalin, then embedded in paraffin and sectioned on a microtome. Alternatively, the brain can be frozen, then sectioned on a cryostat. Brain sections prepared thus can then be analysed for the expression of the SCIP gene, e.g. by staining immunohistochemically, or by in situ hybridisation.

The present invention also provides a kit for detecting SCIP expression comprising a first antibody molecule having affinity for SCIP protein, a second labelled antibody molecule having affinity for the first antibody molecule, development reagents to develop a colour reaction when in combination with the label of the second antibody, appropriate buffer diluents and a counterstain to stain the cells and/or tissue and provide contrast to SCIP containing material labelled using the antibody molecules.

The present invention also provides a further kit for detecting SCIP expression by in situ hybridisation (ISH), wherein the kit comprises a labelled nucleic acid probe encoding a sequence complimentary to SCIP mRNA, buffered solutions for preincubation and incubation steps, a labelled antibody molecule having affinity for the labelled nucleic acid probe, development reagents which develop a colour reaction on contact with the labelled antibody molecule, appropriate buffered diluents and a counterstain to stain the cells and/or tissue and provide contrast to SCIP containing material which is labelled using the labelled nucleic acid probe and antibody molecule.

It is further preferred that the kits of the present invention comprises suitable components for performing a negative and/or a positive result. The components for performing a positive results are used to detect a gene expressed in the tissue of interest.

It could be a constitutively expressed gene, such as GAPDH, or a tissue-specific gene, which in the nervous system could be neurofilament, tau, or glial fibrillary acidic protein. The negative results is preferably obtained by using a nucleotide probe having the sequence of the SCIP gene itself. This is a standard approach known by those practiced in the art.

As indicated above the kit for detecting SCIP expression using an antibody molecule comprises:

A first antibody molecule having affinity for SCIP protein.

A second antibody molecule having affinity for the first antibody molecule. Usually the second antibody molecule is an antibody raised in a second species that specifically reacts to immunoglobulins of the species in which the first antibody molecule was raised. The second antibody molecule preferably has conjugated to it either a fluorescent or enzyme label, as is conventional for indirect immunohistochemistry. Examples of fluorescent labels are FITC or RITC: examples of enzyme labels are a HRP or alkaline phosphatase.

Development reagents. These are used to develop a colour reaction when in contact with the label of the second antibody molecule. Examples are diamino-benzidine and hydrogen peroxide for peroxidase-linked conjugates. These are provided with appropriate buffered diluents.

Diluents for both the first and second antibody molecules typically comprise a buffered saline solution plus a source of protein, e.g. bovine serum albumin, plus a detergent, e.g. Triton-X100.

Counterstains, to stain the cells and/or tissue and provide contrast to the SCIP-stained material are well known to those skilled in the art.

As indicated above the kit for detecting SCIP expression by ISH comprises:

a nucleic acid probe encoding sequences identical to and complimentary with SCIP mRNA. These probes will typically carry a label such as a hapten, e.g. digoxygenin, for subsequent detection.

A number of buffered solutions for the various pre-incubation and incubation steps in the procedure.

An labelled antibody molecule having affinity for the labelled nucleic acid, e.g. an anti-digoxygenin antibody, conjugated to a label, such as alkaline phosphate. A diluent for this antibody molecule is also preferably included.

Development Reagents. Enzyme reagents are generally used which develop a colour reaction, on which the detection is based. Examples are NBT (4-nitro-blue tetrazolium chloride) and BCIP (5-bromo-4-chloro-3-indolyl phosphate) diamino-benzidine and hydrogen peroxide for peroxidase-linked conjugates. These are provided with appropriate buffered diluents.

A counterstain, to stain the cells and/or tissue and provide contrast to the SCIP-stained material.

The present invention allows any nerve cells and/or tissue that are expressing SCIP to be visualised by standard microscopy. The pattern of expression can then be compared with control animals (e.g. adult rats or mice of over 40 weeks of age) or humans, and areas of the tissue identified where SCIP is being expressed specifically in the areas of damage. By virtue of this identified SCIP expression, practitioners will be readily able to determine whether the subject has neurological damage. They will also be able to ascertain which precise area of the nervous system has been adversely affected. This allows conclusions to be drawn concerning the damage to the nerve cells and/or tissue by the disease or the experimental manipulation to which the subject has been subjected.

In neurotoxicology, the present invention provides a quick and accurate means of identifying neurotoxic agents. It is useful for the assessment of novel drugs or in toxicological screens of other compounds, such as assessments of potentially toxic environmental agents or bacterial toxins.

In neuropathology, the present invention provides a quick and accurate means of identifying the nature and location of neuropathology associated with those diseases where specific markers of neuropathology are not available. This invention can be used as a diagnostic for subjects that are alive orpost-mortem or to investigate the pathology of different neurological disorders.

The present invention is now illustrated in the appended examples with reference to the following figures.

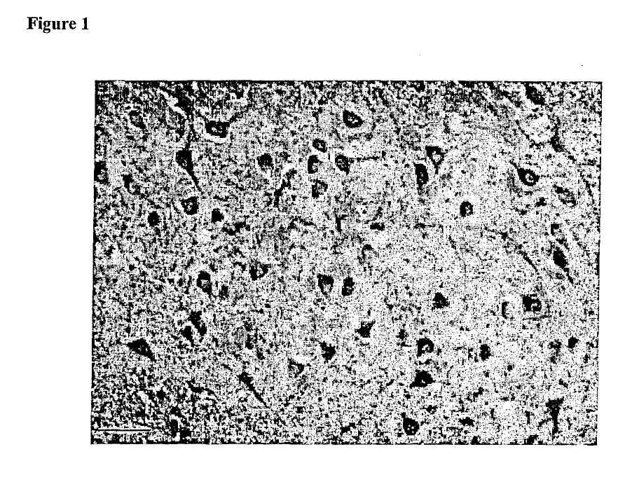

FIG. 1 shows SCIP staining in the CA4 region of the hippocampus. Scale bar: 50 μm.

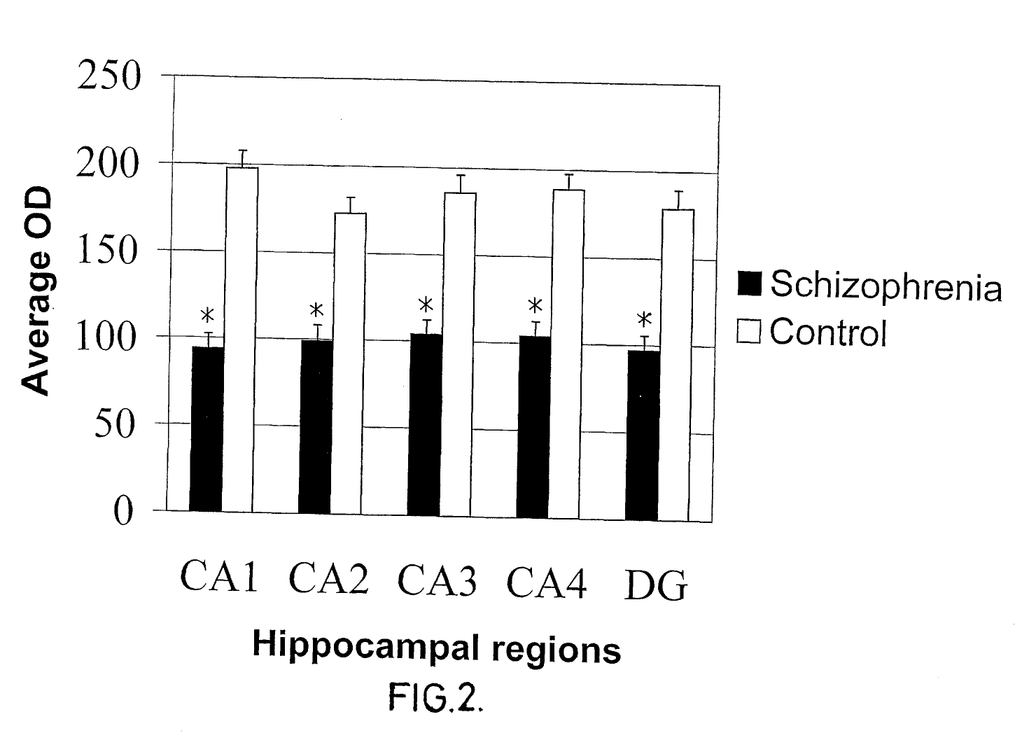

FIG. 2 shows the mean optical density of SCIP stained neurons in the CA1, CA2, CA3, CA4 and dentate gyrus regions in schizophrenic and control groups.

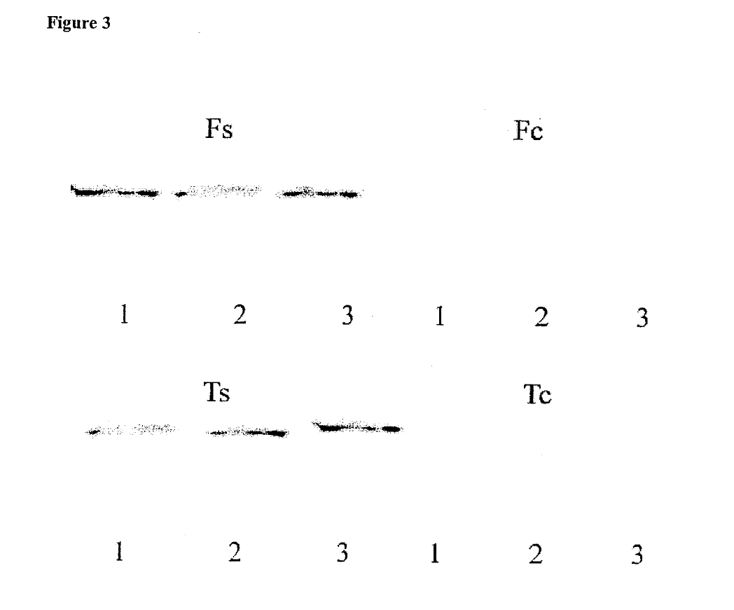

FIG. 3 shows Western blot analysis. Brain extracts from the frontal (Fs) and temporal lobe (Ts) of three schizophrenics were compared with similar brain regions (Fc and Tc) of matched controls using a polyclonal antiserum against SCIP. SCIP was recognised as a 45 KDa product.

### EXAMPLES

Materials and Methods

Tissue Preparation

Human Tissue

Surgical samples were collected either from MRC Brain Bank, Institute of Psychiatry, King's College London, or acutely from surgical specimens. The demographic characteristics of the samples used in Example 1 are described in Tables 1 and 2. There were no significant differences in age, gender or post-mortem interval between groups (Table 3). Exclusion criteria covered any central nervous system related disorders such as head injury, alcohol dependence or Alzheimer's disease. Tissue was obtained from patients with a clinical diagnosis of schizophrenia according to DSM-III-R criteria. Mean neuroleptic exposure in the month prior to death was estimated for schizophrenic subjects and expressed in chlorpromazine equivalents (CPZE).

Separate tissue specimens were also obtained from patients with a pathological diagnosis of either focal cortical dysplasia or Alzheimer's disease.

The tissue preparation was standard for histopathological specimens. The specimens were fixed in 10% formalin for between 24-48 hours, cut into between 4 and 20 slices depending on the size of the specimen, then embedded in paraffin blocks and sectioned at 7 μm.

Rodent Tissue

Tissue specimens were taken from BalbC mice over 40 weeks of age that had undergone unilateral brain injury in the hippocampal region, and from Wistar rats with induced global ischaemia. The tissue specimens were fixed in 4% paraformaldehyde overnight at 4° C., embedded in paraffin wax and sectioned at 7 μm.

Neurotoxic Injury

Adult rats or mice were injected intra-peritoneally with a compound known to cause neurotoxic effects, for example, phenytoin (75 mg/kg) or 3-nitropropanoic acid (120 mg/kg). One day following this injection, the animals were killed using standard approved techniques, and their brains were removed and processed for immunocytochemistry. This preparation is a standard procedure for those knowledgable in the art. It involves fixation of the tissue with 4% paraformaldehyde, cryoprotecting the tissue by immersion overnight in 30% sucrose solution, then freezing of the tissue in liquid nitrogen. The tissue is then cut on a cryostat at a thickness of 10 μM. The tissue sections are then processed for immunocytochemistry using standard procedures.

Preparation of Antibody

The tissue sections are stained using an antibody that reacts specifically with the protein, SCIP. The antibody can be prepared according to the method of Meijer et al., Nucleic Acids Res., 18, 7357-7365 (1990); Meijer et al., Nucleic Acids Res., 20, 2241-2247 (1992). Typically, such an antibody can be raised against a purified preparation of the protein prepared by over-expression of the protein in E. coli, into which has been introduced an expression plasmid encoding SCIP. This can be achieved by cloning the BamHI-BglII fragment from pN1SCIP behind the Isopropyl β-D-Thiogalactopyranoside (IPTG) inducible T7 promoter in the BamHI site of the pET11A expression vector (Novagen). See Meijer et al., Nucleic Acids Res., 18, 7357-7365 (1990); Meijer et al., Nucleic Acids Res., 20, 2241-2247 (1992). This construct can then be transfected into the BL21 strain of E. coli. An overnight culture is diluted 1 in 10 and cultured at room temperature to an OD₆₀₀=0.8. Over-expression is induced by adding IPTG to a final concentration of 0.4 mM and the culture is incubated for 4 hours.

For large scale purification, a 500 ml IPTG induced bacteria culture is pelleted, washed once with Phosphate-Buffered Saline (PBS), resuspended in 10 ml 6M urea/PBS and sonicated. The cell lysate is cleared by centrifugation at 12000 rev./min for 5 min at 4° C.

Imidazole is added to the cell lysate to a final concentration of 0.8 mM and incubated overnight at 4° C. with 300 μl Ni-NTA beads (Qiagen). The following day, the Ni-NTA is washed twice with 10 ml of a 6 M urea/PBS/80 mM imidazole solution for 15 min and three times with 6 M urea/PBS/8 mM imidazole solution. SCIP protein is eluted from the matrix in 500 μl 6 M urea/PBS/0.8 mM imidazole solution. This purification procedure produces high levels of pure (>95%) and intact SCIP protein as judged by Coomassie stained polyacrylamide gel electrophoresis (SDS-PAGE). See Zwart et al., Mech. Dev., 54, 185-194 (1996).

Generation of Anti-SCIP Antiserum

Following over-expression and purification of the SCIP protein, antibodies can be raised in rabbits (White New Zealand) by three consecutive injections of 0.5-1.0 mg SCIP protein resuspended in Freund's adjuvant with a 4 weeks interval between each injection. See Zwart et al., Mech. Dev., 54, 185-194 (1996).

SCIP antibodies are then affinity purified by binding to the SCIP protein immobilised on nitrocellulose. After preincubation with 1% BSA/3% powdered milk/0.05% Tween-20/PBS for 2 h at 4° C., the nitrocellulose is incubated overnight with the antiserum that has been precleared with BL21 cell lysate at room temperature for 3 h. After extensive washing with PBS the SCIP antibodies are eluted from the nitrocellulose by 3 M KSCN/0.1 M NaPO₄/500 μg/ml BSA solution. To remove the KSCN the antibody solution is passed over a 0.1 M NaPO₄ (pH 7.5) equilibrated Sephadex G-50 column. See Zwart et al., (supra).

The SCIP polyclonal antiserum raised by this method is highly specific since it does not cross react with other POU proteins such as Oct-1/3/4, Bm-1/3/4. In addition to this, there is great homology of isolated SCIP cDNA between human and rodents with the human sequence (Tobler et al., Nucleic Acids Res., 21, 1043 (1993) being 98.8% homologous to the sequence of mice (Zimmerman et al., Nucleic Acids Res., 19, 956 (1991) and rats (He et al., Nature, 340, 6228 (1989); Monuki et al., Science, 249, 1300-1303, (1990)). The antibody can be used to detect rodent and human SCIP protein in immunohistochemical applications.

Immunohistochemistry

The sectioned brain material was stained immunohistochemically to reveal the presence and location of immunoreactive SCIP in the tissue section. This was done using standard immunohistochemical procedures.

Wax-imbedded sections were dewaxed and rehydrated in methanol. Frozen sections were kept at −20° C., and brought to room temperature immediately before use. Thereafter the procedure for both types of material was the same. To block non-endogenous peroxidase activity, the sections are incubated with methanol/3% H₂O₂ solution for 20 min. After extensive washes first with distilled water and then with Tris-Buffered Saline (TBS), the sections are blocked with normal swine serum (Dako), diluted 1:10 in TBS, for 30 min at room temperature and then incubated in the primary anti-SCIP (1:250) antibody in TBS overnight at 4° C.

For bright-field microscopy, sections are incubated for 45 min with a biotinylated Swine anti-Rabbit secondary antibody at 1:200 (Dako) and then for 45 min with an avidin-biotin-peroxidase complex (Vector Laboratories), followed by a 5 min reaction with a diamino benzidine (DAB)/0.03% hydrogen peroxide in PBS kit (Vector Laboratories). The samples are then dehydrated in an ethanol series, followed by three rinses in xylene, and then permanently mounted with DPX mounting medium and coverslipped.

For fluorescence microscopy, immunolabelled sections are incubated for 1 h at room temperature with rabbit conjugated fluorescent markers at 1:200 (Vector). Sections are then embedded in anti-fade media (Vectashield) and coverslipped for storage.

Following the staining procedure, SCIP expression can be detected by light and/or fluorescent microscopy. Cells in the tissue sections that were expressing SCIP will be labelled by the antibody staining procedures. In normal undamaged adult brain material, such cells are rare. This is an indication that the damage induced SCIP expression, and that the SCIP immunoreactivity is diagnostic of the damage, and that the sites of SCIP immunoreactivity are indicative of the sites of damage.

In situ Hybridisation

SCIP expression can be detected using in situ hybridisation (ISH) rather than immunohistochemistry. In this case, the presence of mRNA encoding the SCIP protein is detected rather than the protein itself. ISH is a standard technique familiar to those practiced in the art (Wilkinson, D. G., In Situ Hybridisation: A Practical Approach, 1st Edn, 87-106, 1992). The sections from damaged brain material are dewaxed in Histoclear three times for 10 min each, followed by 2 washes in methanol for 5 min each. Then, sections are rehydrated through a graded series (100%, 75%, 50% and 25%) of methanols made up in PBT for 5 min each and washed twice with PBT for 5 min each. After rehydration, sections are treated with 10 μg/ml proteinase K (Boehringer Mannheim) in PBT for 10 minutes at 37° C.; refixed in 4% parafomaldehyde in PBS for 20 min and acetylated with 0.1 M triethanolamin acetate. Slides are then dehydrated via 25%, 50%, 75% and 100% series of methanol for 5 min in each. To block non-specific binding of RNA probes, sections are prehybridized with a buffer containing 5×SSC (0.3 M NaCl, 0.03 M sodium citrate, pH 7.4), 50% deionized formamide (BDH), 1 m g/ml yeast tRNA (Boehringer Mannheim), 5 mM EDTA, 50 μg/ml heparin, 0.1% tritonX-100, 0.5% CHAPS (Sigma) and 2% blocking reagent (Boehringer Mannheim). The sections are prehybridised at 56° C. for 2 hr. The hybridization buffer is made up by adding cRNA probe (final concentration 5×10⁶ cpm/ml) to the prehybridization buffer. Sections are covered with the hybridization buffer and incubated in a sealed humid box at 60° C. overnight. Following hybridization, slides are washed twice with 2×SSC/50% formamide at 60° C. for 30 min and treated with RNAse (20 μg/ml) at 60° C. for further 30 min. After extensive washes in 2×SSC and 0.2×SSC for 15 min each at 37° C., the hybridised sections are blocked in 5% goat serum (Sigma) for 1 hr and incubated overnight at 4° C. in alkaline phosphatase-conjugated sheep anti-DIG diluted 1:3500 (Boehringer Mannheim). The following day, sections are washed with NTMT buffer made of 100 mM Tris-HCl pH 7.5, 50 mM MgC_{12, 100} mM Nacl and 0.1% triton X-100, and incubated in the colour reagent NBT/BCIP (Boehringer Mannheim) until sufficient signal has developed. The signal is then fixed by immersing the slides in 4% paraformaldehyde. The sections are then counterstained with cresyl violet (Nissl) and dehydrating via 25%, 50%, 75% and 100% series of methanol for 5 min in each, followed by Histoclear and coverslipped with mounting media DPX (13DH).

The RNA probes for the SCIP mRNA are already in the public domain (Suzuki et al., EMBO, 11, 3723-3732 (1990): Zwart et al., Mech. Dev., 54, 185-194 (1996). The RNA probe is preferably a 160 bp of the mouse SCIP cDNA fragment 5′ggaggcggcggcgcgggacccggcctgcaccacgcactgcacgaggacggccacgaggcacactggagccgtcgccaccaccgcacctgggcgcacacggacacgcacggacatgcacacgcgggcggcctgcacgcggcggcggcggcgcacctgcaccggg3′), subcloned into Bluescript (Stratagene) and linearised with the restriction endonuclease SmaI. RNA probes can then be transcribed using T3 and T7 polymerases according to the manufacturer's instructions (Promega). Expression patterns are visualised using digoxygenin (DIG)-UTP-labelled sense and antisense RNA probes and anti-DIG antibodies conjugated to alkaline phosphatase (Boehringer Mannheim).

As with the immunohistochemical detection, the expression of SCIP can be detected by this method. Thus it will be apparent that SCIP expression has been upregulated at sites of neurological damage, and is thus a marker of those sites of damage.

### EXAMPLE 1

Analysis of Brain Tissue from Patients with Alzheimer!S Disease

Based on the methods described above blocks of temporal lobe were taken at the level of the lateral geniculate body and included the parahippocampal gyrus and hippocampus. Blocks of the frontal lobe were taken at the level of the sharp ventral curve at the anterior end of the corpus callosum trunk. The subjects from which the samples are taken are shown in Table 2. All blocks used for immunohistochemistry were fixed in 10% formalin and subsequently coronally sliced before being embedded in paraffin wax.

Seven μm thick sections were stained using standard immunohistochemical procedures to reveal the presence and location of SCIP protein. Briefly, sections were dewaxed, rehydrated in methanol and pre-treated with 1% H₂O₂ for 30 minutes. Sections were then microwaved at 800 W for eight minutes in a 0.001% solution of citric acid/phosphate buffer (pH 6.0). After extensive washes with Tris-Buffered Saline (TBS), the sections were blocked with normal swine serum (Dako), diluted 1:10 in TBS, for 30 min and then incubated in the primary rabbit polyclonal anti-SCIP(1:250) antibody in TBS overnight at 4° C. The SCIP polyclonal antiserum used in this study was raised against the N-terminal region of SCIP, a region of least homology with other POU proteins such as Oct-1/3/4 and Bm-1/3/4. The three-step avidin-biotin-horse-radish peroxidase complex system was used (Dako, Ltd) and the antibody was visualised using the chromogen diaminobenzidine (Vector). Negative controls consisted of duplicate sections that were processed in parallel and consisted of adjacent tissue sections in which the primary antibody was replaced by TBS.

Western Blot

Protein extracts were prepared from the temporal and frontal lobes of three schizophrenic and three control cases. Each extract was washed twice with PBS and lysed by the addition of 1% Nonidet P40 lysis buffer (0.5 M Tris-HCl pH 8.0, 3 M NaCl, 0.5M EDTA plus protease inhibitors: 2 μg of pepstatin per ml, 2 μg of leupeptin per ml, 1 μg of peprotonin per ml) and vortexing. Solubilised samples were then centrifuged at 13,000 rpm at 4° C., for 10 min. The protein concentration from each extract was estimated by performing a DC protein assay (BioRad). After protein quantification, samples were solubilised in standard sodium dodecyl sulfate (SDS) sample buffer (0.25M Tris-HCl pH 6.8, 0.2% bromophenol blue, 40% glycerol, 20% 2-mercaptoethanol and 8% SDS), denatured, loaded on 10%Tris-Polyacrylamide gels (BioRad) and run at a constant 200 Volts for 35 minutes. The proteins were then transferred to 0.2 μm nitrocellulose paper (Sigma) using a semidry blotting apparatus (BioRad) and run at 10 Volts for 30 minutes. The blots were blocked with 10% casein solution (Sigma) for 30 min and they were then treated with avidin C/biotin kit according to the manufacturer's instructions (Sigma). Next, the membranes were washed with TBS-T (25 mM Tris-HCl pH 7.5, 0.5 M NaCl and 0.3% Tween 20) and incubated with primary polyclonal antibody anti-SCIP(1:3500) in TBS-T for 30 minutes. Blots were washed with TBS-T and incubated with secondary biotinylated goat anti-rabbit antibody (Vector) for 30 minutes. Finally, a Vectastain ABC complex system was used (Vector) and the blots were treated with the chromogen diaminobenzidine (Vector) until bands could be clearly seen. Negative controls consisted of duplicate blots that were processed in parallel in which the primary antibody was replaced by TBS-T.

Image Analysis

All sections were analysed using a Leica light microscope with image analysis software (Image Pro-plus) and motorised stage. This system enabled us to tie together separate microscopic fields, viewed individually at high magnification to form single composite images of large strips encompassing the hippocampal formation. The boundaries of the hippocampal formation were drawn at low magnification and each subregion delineated using standard criteria described previously (Lorento de No, J. Psychiatry Neurol., 1934; 46: 113-177; Amaral D G, Insausti R: Hippocampal formation. In Paxinos G. (Ed.), The Human Nervous System. Academic Press 1990; 711-756). In order to randomly select neurons for each of the five regions, the image of the hippocampal composite was captured and a grid of crosses was placed on top of it.

The optical density of SCIP stained neurons was quantified in the CA1, CA2, CA3, CA4 and dentate gyrus (DG) regions for both schizophrenic and control cases using a 256-point grey scale. For the schizophrenic cases, the cytoplasmic staining of neurons whose nuclei were visible in section were analysed. For the control cases, there was sufficient background staining to enable us to identify the cytoarchitecture of the hippocampus and make comparable cytoplasmic analysis of neurons. Optical density readings were estimated only for neurons that were intersecting with the crosses of the grid. The mean optical density values across the fields of each region were then calculated.

Data were analysed using the Mann Whitney U rank sum test (SPSS 10.0). To adjust for multiple comparisons the Bonferroni correction factor was applied, and a p value of 0.01 was considered significant.

Results

Immunohistochemical Staining

SCIP was widely expressed in the hippocampus of all schizophrenic specimens whilst there was little or no staining above background in the control cases. SCIP staining was predominantly cytosolic and it was seen in the pyramidal cell layer of the hippocampus and in the granule cell layer of the dentate gyrus (FIG. 1). In the temporal lobe of schizophrenic samples, SCIP staining was more prominent in the CA2, CA3, CA4, and in the granule cell layer of the dentate than staining in the CA1. No similar conclusions could be drawn for the matched control sections as there was no or very little SCIP immunoreactivity present.

To assess the intensity of SCIP staining in the schizophrenic and control samples, the optical density patterns were quantified in the CA1, CA2, CA3, CA4, and in the dentate gyrus. FIG. 2 shows mean optical density estimates per hippocampal subregion for control and schizophrenic samples. Mann Whitney U rank tests revealed that there were significant reductions in optical density measurements in the schizophrenic group in all hippocampal regions examined, with p values being less than 0.001 in all cases between schizophrenics and controls. This shows that the intensity of SCIP staining was significantly higher in the schizophrenic subjects than in the controls.

To explore the possibility that neuroleptic medication, age of subjects and/or postmortem delay may affect the expression of SCIP, the correlation of each of the above factors with the mean optical density values obtained for each of the 5 regions using Spearman's rank correlation test was analysed. In the schizophrenic group, there was no significant correlation between SCIP staining and mean neuroleptic exposure (CPZE) (p>0.1 in all regions), neither was a significant relationship found between SCIP staining and age or post-mortem delay in any regions (p>0.1 in all cases).

Western Analysis

Protein levels of SCIP were examined in extracts from the frontal and temporal cortex of three schizophrenics and three matched controls. Immunoblots confirmed that the SCIP antibody recognises a single protein of about 45 KDa, as expected. There were high levels of SCIP in the frontal and temporal lobe of the schizophrenic specimens whilst there was no or very little SCIP expression in the same regions of the matched controls (FIG. 3).

The results demonstrates that extensive SCIP immunoreactivity is present in the frontal and temporal lobes of schizophrenic specimens, whilst there is limited expression of SCIP in matched controls. The findings indicate that SCIP is useful as a neuropathological marker in schizophrenia as well as a marker of any neurological damage.

Testing of Compounds for Neurotoxicity

The neurotoxicity of compounds can be tested according to the invention by contacting cells, tissues or animals with test compounds and testing for the expression of SCIP by methods described above. Increased levels of SCIP expression are indicative of neurotoxicity and therefore compounds which do not lead to neurotoxicity are selected. Methods of contacting cells, tissue or animals are well known to those skilled in the art.

All references referred to herein are hereby incorporated by reference.

TABLE 1
Cases used for the temporal lobe immunohistochemical study

| | | | Diag- | | PM | |
|---|---|---|---|---|---|---|
| Case | Age | Gender | nosis | CPZE | delay | Cause of death |
| 1 | 24 | M | S | 200 | 29 | Renal failure |
| 2 | 34 | M | S | 4000 | 21 | Myocarditis |
| 3 | 46 | F | S | 600 | 96 | Cardiac arrest (OD) |
| 4 | 49 | M | S | 700 | 25 | Ruptured aneurysm |
| 5 | 62 | M | S | 350 | 31 | Peritonitis |
| 6 | 68 | M | S | 200 | 45 | Myocardial Infarction |
| 7 | 73 | M | S | 0 | 25 | Pneumonia |
| 8 | 74 | M | S | 3500 | 23 | Myocardial Infarction |
| 9 | 75 | M | S | 500 | 94 | Pneumonia |
| 10 | 88 | F | S | 0 | 20 | Pneumonia |
| 11 | 20 | M | C | — | 26 | Multiple injuries |
| 12 | 33 | F | C | — | 96 | Pulmonary embolus |
| 13 | 44 | M | C | — | 70 | Myocardial infarction |
| 14 | 51 | M | C | — | 15 | Pneumonia |
| 15 | 63 | M | C | — | 26 | Coronary artery |
| | | | | | | occlusion |
| 16 | 64 | M | C | — | 47 | Myocardial infarction |
| 17 | 76 | M | C | — | 41 | Bronchopneumonia |
| 18 | 80 | F | C | — | 31 | Pulmonary embolus |
| 19 | 80 | M | C | — | 35 | Left ventricular failure |
| 20 | 86 | M | C | — | 6 | Myocardial infarction |

CPZE: mean daily neuroleptic exposure a month prior to death, in chlorpromazine equivalents;
S: schizophrenia;
C: control;
PM post-mortem

TABLE 2
Cases used for frontal and temporal lobe Western analysis.

| | | | | | PM | |
|---|---|---|---|---|---|---|
| Case | Age | Gender | Diagnosis | CPZE | delay | Cause of death |
| 21 | 32 | F | S | 500 | 46 | Pulmonary |
| | | | | | | embolus |
| 22 | 51 | M | S | 800 | 44 | Myocardial |
| | | | | | | Infraction |
| 23 | 62 | M | S | 300 | 36 | Pulmonary |
| | | | | | | tuberculosis |
| 24 | 33 | F | C | — | 56 | Pulmonary |
| | | | | | | embolus |
| 25 | 51 | M | C | — | 52 | Chronic |
| | | | | | | cardiomyopathy |
| 26 | 67 | M | C | — | 41 | Myocardial |
| | | | | | | Infraction |

CPZE: mean daily antipsychotic exposure a month prior to death, in chlorpromazine equivalents;
S: schizophrenia;
C: control;
PM: post-mortem

TABLE 3
Comparison of demographic factors in schizophrenia and
control groups used in the temporal lobe study and in the
frontal versus temporal lobe study

| | | | Frontal & Temporal | |
|---|---|---|---|---|
| | Temporal lobe study | | lobe study | |
| | Schizo- | | Schizo- | |
| | phrenia | Control | phrenia | Control |
| Age (years) | 59.3 (20.3) | 59.7 (22.2) | 483 (15.2) | 50.3 (17.1) |
| Gender | 8M/2F | 8M/2F | 2M/1F | 2M/1F |
| (M/F) | | | | |
| Post-mortem | 40.9 (29.3) | 39.3 (26.6) | 42 (5.3) | 49.6 (7.8) |
| delay | | | | |

Values are mean and (standard deviation)

1. A method of detecting neurological damage comprising assaying for the expression of a SCIP gene in nerve cells and/or tissue.

2. The method of claim 1, comprising assaying for the presence of SCIP protein.

3. The method of claim 2, wherein a immunohistochemical assay is used to detected the presence of SCIP protein

4. The method of claim 2 or claim 3, comprising obtaining a sample of nerve cells and/or tissue from a subject and contacting the nerve cells and/or tissue with an antibody molecule having affinity for SCIP protein in order to determine if SCIP protein is present.

5. The method of claim 4, wherein the antibody molecule is a monoclonal antibody.

6. The method of claim 4 or claim S, wherein the antibody molecule is labelled.

7. The method of claim 6, wherein the antibody molecule is labelled with horseradish peroxidase, chloramphenicoltransferase, digoxygenin, fluorescein or a radioisotopes.

8. The method of claim 4 or claim 5, Wherein the antibody molecule is detected by a labelled antibody molecule having affinity for the antibody molecule having affinity for SCIP protein.

9. The method of claim 1, comprising assaying for the presence of SCIP mRNA.

10. The method of claim 9, wherein an in situ hybridisation assay is used to detect the presence of SCIP mRNA.

11. The method of claim 9 or claim 10 comprising obtaining a sample of nerve cells and/or tissue from a subject and contacted the nerve tissue with a probe that specifically recognises SCIP mRNA.

12. The method of claim 11, wherein the probe is labelled.

13. The method of claim 12, wherein the probe is labelled with digoxygenin.

14. The method of any one of claims 11 to 13, wherein the probe is a nucleic acid probe.

15. The method of claim 14, wherein the nucleic acid probe is a DNA or an RNA probe.

16. The method of any one of claims 14 to 15 wherein the probe is about 10 to 500 nucleotides in length.

17. The method of any one of claims 14 to 16, wherein the probe has a sequence corresponding to that of at least part of the SCIP mRNA.

18. The method of claim 17, wherein the sequence of the probe corresponds to any part of the SCIP mRNA which is unique to the SCIP mRNA.

19. The method of claim 18, wherein the sequence of the probe corresponds to part of the SCIP mRNA encoding the N-terminal region of the SCIP protein.

20. A kit for detecting SCIP in nerve cells and/or tissue expression comprising a first antibody molecule having affinity for SCIP protein, a second labelled antibody molecule having affinity for the first antibody molecule, development reagents to develop a colour reaction when in combination with the label of the second antibody, appropriate buffer diluents and a counterstain to stain nerve cells and/or tissue and provide contrast to SCIP containing material labelled using the first and second antibody molecules.

21. The kit of claim 20 additionally comprising one or more components for obtaining a negative and/or a postive result.

22. A kit for detecting SCIP expression in nerve cells and/or tissue by in situ hybridisation (ISH), wherein the kit comprises a labelled nucleic acid probe encoding a sequence complimentary to SCIP mRNA, buffered solutions for preincubation and incubation steps, a labelled antibody molecule having affinity for the labelled nucleic acid probe, development reagents which develop a colour reaction on contact with the labelled antibody molecule, appropriate buffered diluents and a counterstain to stain nerve cells and/or tissue and provide contrast to SCIP containing material which is labelled using the labelled nucleic acid probe and antibody molecule.

23. The kit of claim 22 additionally comprising one or more components for obtaining a negative and/or a postive result.

24. A method for detecting schizophrenia in a subject comprising assaying for increased levels of SCIP expression in cells of the subject by a method according to any one of claims 1 to 19 or using a kit according to anyone of claims 20 to 23.

25. A method for assaying neurotoxicity of a test compounds comprising contacting nerve cells and/or tissue with a test compound and assaying for SCIP expression in nerve cells and/or tissue.

26. The method according to claim 25, wherein the nerve cells and/or tissue are contacted with the test compound in vitro.

27. The method according to claim 25, wherein the nerve cells and/or tissue are contacted with the test compound in vivo.

28. The method according to claim 27, wherein the test compound is given to an animal.

29. The method according to any one of claims 25 to 28 in which increased levels of SCIP expression in the nerve cells and/or tissue indicates neurotoxicity of the test compound.
